(19) United States
(12) Patent Application Publication
(10) Pub. No.: US 2011/0009789 A1
(43) Pub. Date: Jan. 13, 2011

(54) Cervical Spine Immobilization Device

(76) Inventor: Stephen W. Barns, Granville, OH (US)

Correspondence Address: MILLER & BARNS, PLLC, 230 EAST BROADWAY, SUITE 250, GRANVILLE, OH, 43023, US

(21) Appl. No.: 12/499,360

(22) Filed: Jul. 8, 2009

(51) Int. Cl. A61F 5/00

(52) U.S. Cl. 602/17; 602/18

(57) ABSTRACT

The invention provides a cervical spine immobilization device that is deployed in the field to contact both sides of the head as well as the jaw, neck and/or cervical collar. The device may be formed in the shape of a polyhedron with a number of cuts to form articulated links or extensible sections. The extensible sections form at least two articulated blocking sections that may be positioned on either side of the head. There may also be multiple extensible sections to form four blocking sections, two of which may be arranged on either side of the head and two that are articulated to compress against the jaw, cervical collar or neck of a patient. The extensible sections may also be easily torn to allow removal of blocking sections to improve patient access or to fit the device to the patient. The device provides a disposable restraint that provides secure immobilization and is adjustable to fit nearly any head size. The cervical spine immobilization device also provides firm restraint during transport of athletes wearing helmets.

### BACKGROUND

Cervical spine immobilization devices have been used for many years by emergency medical technicians. To secure a patient having a suspected spinal injury to a back board. After trauma such as motor vehicle crashes, falls and sports injuries where spinal injury is suspected, patients are secured to a spine board using webbing across the chest, stomach and knees. The neck is immobilized using a cervical collar and the head is secured to the board using a head immobilization device. The patient is then transported to and emergency department for further evaluation and care. Secure immobilization is important in preventing additional injury, which may lead to paralysis.

Head immobilization devices fall into two general categories. Reusable head immobilization devices are typically formed of a vinyl coated foam blocks and may be washed following use. Recently, these reusable devices have fallen out of favor due to the effort required to clean blood and other bodily fluids from the pads and the high replacement cost. Typically, the patient is left at the hospital or air evacuated with the head immobilization device in place. Often the device is not returned. In response to these drawbacks, disposable head immobilization devices have been developed. These disposable devices are typically formed of cardboard, are substantially less expensive and do not require cleaning. However, it is believed that some degree of security and immobilization is lost.

One typical reusable head immobilization device is shown in U.S. Pat. No. 6,659,104 which shows a lower pad fixed to the spine board and having hook and loop fasteners the lower pad. The '104 patent also includes two lateral pads which are affixed to lower pad by the hook and loop fasteners and are retained against the head by adjustable straps. Many other similar cervical spine immobilization devices are available; the most common of these is sold by FERNO-Washington Inc. (Wilmington, Ohio, USA) under the trademark Universal Head Immobilizer.

Disposable cervical spine immobilization devices are available in a number of configurations. U.S. Pat. Nos. 6,158,819 and 6,176,549 disclose a foldable cardboard head rests that form triangular shaped support members on opposite sides of the patient's head. U.S. Pat. No. 5,146,641 discloses a cervical spine immobilization device with flaccid loops that are designed to receive compressible blocks to form triangular support members. U.S. Pat. No. 6,966,321 discloses a complex head immobilization device that includes a head harness, a forehead strap, a chin strap, a crown strap and a lateral stabilization strap. U.S. Pat. Nos. 6,913,584; 6,398,747 and 7,297,127 disclose a head immobilization device that includes a pair of arms that may be folded upward by pulling on an actuating device. The actuating device is linked to the outer ends of the arms for folding the arms symmetrically and uniformly. U.S. Pat. No. 5,211,185 discloses restraint having a base portion which is adhered to the spine board and a flexible portion that encircles the head with a band of material.

Typically, these cardboard restraints are secured to the board with a double faced adhesive prior to positioning the patients. Securing the restrain to the board poses a number of problems. Typically, it is desired to position the patient on the board and subsequently position the cervical spine immobilization device without lifting or moving the patient's head. Pediatric patients are often immobilized in a car safety seat, rather than removing the child from the seat. The double faced adhesive does not adhere to the material of the safety seat and other materials must be used. Patients with hyperkyphotic (head forward) posture, which is often accompanied by an upper thoracic hump, cannot be laid flat on a spine board and must be supported, typically with blankets. Cardboard restraints using double faced adhesive are typically not suitable for hyperkyphotic patients.

Disposable foam restraints have also been developed to limit head and neck movement. U.S. Pat. No. 6,088,855 discloses a pediatric cervical neck support, which is not for use in emergency medical situations, having a semicircular recess cut out from a foam support. U.S. Design Pat. No. D535,920 shows a pad for head and neck restraint that is a generally horseshoe shaped pad. U.S. Pat. No. 4,528,981 teaches two foam cylinders with a flexible connection to allow the cylinders to roll in toward one another to contact the patients head. These foam restraints typically do not conform to the head and do not contact the patients head consistently.

### SUMMARY

In accordance with embodiments of the present invention, the present invention relates to an articulated cervical spine immobilization device which conforms to the head. The cervical spine immobilization device of the present invention includes a unitary construction with extensible sections to allow the cervical spine immobilization device to be stored compactly. The cervical spine immobilization device of the present invention is deployed in the field to contact both sides of the head as well as the jaw, neck and/or cervical collar. In one embodiment, the cervical spine immobilization device is formed in the shape of a polyhedron with a number of cuts to form articulated links or extensible sections. The extensible sections form at least two articulated blocking sections that may be positioned on either side of the head. In another embodiment, there are multiple extensible sections to form four blocking sections, two of which may be arranged on either side of the head and two that are articulated to compress against the jaw or cervical collar. In yet another embodiment of the invention, the extensible sections form easily torn sites to allow removal of articulated blocking sections or extensible sections to improve patient access or to fit the cervical spine immobilization device to the patient. The cervical spine immobilization device may have a generally circular or a polyhedral cross section. The extensible sections may be formed in a molding process, with a second material, or may be formed by a series of cuts across a portion of the cross-section. The cervical spine immobilization device provides a disposable restraint that provides secure immobilization and is adjustable to fit nearly any head size. The cervical spine immobilization device also provides firm restraint during transport of athletes wearing helmets.

In accordance with additional embodiments of the present invention, cervical spine immobilization system is formed of a body having at least one link section dividing the body into a plurality of articulated blocking sections suitable for use in immobilizing a patients head. Generally, the cervical spine immobilization device includes three link sections and four blocking sections two blocking sections contact the cranium to limit motion and two sections contact the jaw and neck or cervical collar to further limit motion. A link section may be included between the two blocking sections that contact the cranium to improve stability and to ease application of the cervical spine immobilization device. Another aspect of the present invention includes separate articulated blocking section without a linking section that may be applied to either side of the head. The cervical spine immobilization device system without the link include two unitary bodies, each of which including two blocking sections and an articulated link therebetween. The articulated sections may be formed in a variety of ways including forming one or more cuts in the material of the blocking sections, one particularly useful link configuration is 3 substantially parallel cuts, two from the exterior of the restrain, as the system is fitted to the patient and one cut from the interior of the restraint to form what is referred to as a W-cut.

### BRIEF DESCRIPTION OF THE DRAWINGS

A more complete appreciation of the invention and the many embodiments thereof will be readily obtained as the same becomes better understood by reference to the following detailed description when considered in connection with the accompanying drawings, wherein:

FIG. 1 illustrates a perspective view of a unitary cervical spine immobilization device of the present invention securing a patient to a spine board.

FIG. 2 illustrates a perspective view of a two piece cervical spine immobilization device of the present invention securing a patient to a spine board.

FIG. 3 illustrates a plan view of the unitary cervical spine immobilization device of the present invention.

FIG. 4 illustrates a plan view of the two piece cervical spine immobilization device of the present invention.

FIG. 5 illustrates plan view of the cervical spine immobilization device showing a generally trapezoidal cross section.

FIG. 6 illustrates a perspective view of a cervical spine immobilization device in accordance with the present invention.

FIG. 7 illustrates a plan view, in detail of an articulated link section usable with the cervical spine immobilization device in accordance with the present invention.

### DETAILED DESCRIPTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

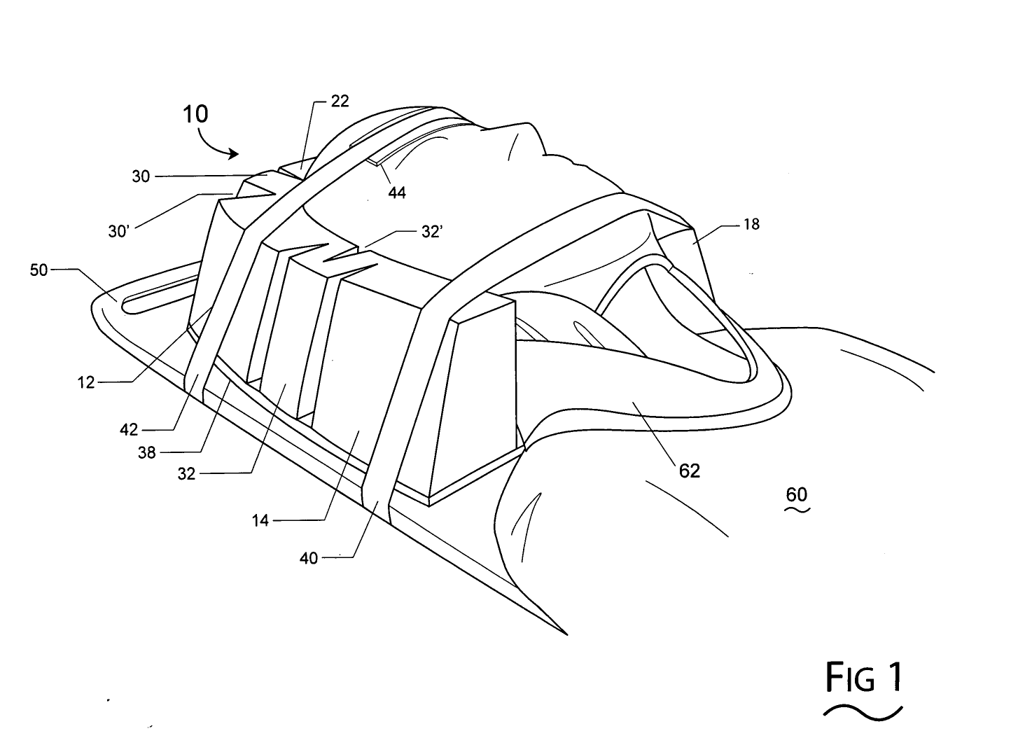

A cervical spine immobilization device, or restraint, 10 according to the present invention is described herein with reference to the figures. As shown in FIG. 1 and FIG. 4, the restrain 10 is typically used in conjunction with a spine board 50 (also known as a back board). At the scene of an injury, a patient 60 is typically fitted with a cervical collar 62, is placed on a spine board 50 and the patient is secured by a number of straps. Once patient 10 is secured to spine board 50 the head is secured to the board using restraint 10 and straps 40, 42.

The restraint 10 may include a blocking section 12 for contacting and restraining the cranium of patient 60 and more specifically temporal bone, mastoid process and/or upper mandible on one side of the patient's cranium. The restraint 10 also includes blocking section 14 for contacting the patient's mandible and/or the cervical collar 62. In the event that patient 10 cannot be fitted with a cervical collar 62, the blocking section 14 may contact the patient's mandible and neck. Blocking sections 12, 14 are connected to one another by an articulated link 32. Articulated link 32 may be formed in a variety of ways such as a flexible sheet or by one or more cuts formed in the body of the restraint 10.

The restraint 10 may include a blocking section 20, opposite blocking section 12 for contacting and restraining the cranium of patient 10. The restraint 10 also includes blocking section 18, opposite blocking section 14, for contacting the patient's mandible and/or the cervical collar 62. Blocking sections 18, 20 are connected to one another by an articulated link 34.

Restraint 10 may also include a central blocking section 22 connected to blocking section 12 and blocking section 20 by articulated links 30, 36. The articulated links 30, 32, 34, 36 may be formed in a variety of ways such as a flexible sheet adhered between the adjacent blocking sections or one or more cuts formed in the body of the restraint 10. FIG. 1 and FIG. 4 shows W-cuts 30′ and 32′ formed by a single cut formed from the interior face 10a of restraint 10 and 2 substantially parallel cuts made from exterior surface 10d of restraint 10. FIG. 4 shows W-cuts 34′ and 36′.

Restraint 10 may also include a head pad 38 to raise the head of patient 60. The head pad 38 may increase patient comfort, and also serves to keep the spine of patient 60 inline during transport and may improve the ability to maintain an open airway. In some cases, a patient with forward head posture (anterior head translation) may require a large head pad 38 to compensate for the curvature of the spine. Often emergency medical personnel will use folded blankets or towels to provide additional padding for the base of the head, in which case, the restraint 10 may be place on the additional padding (not shown).

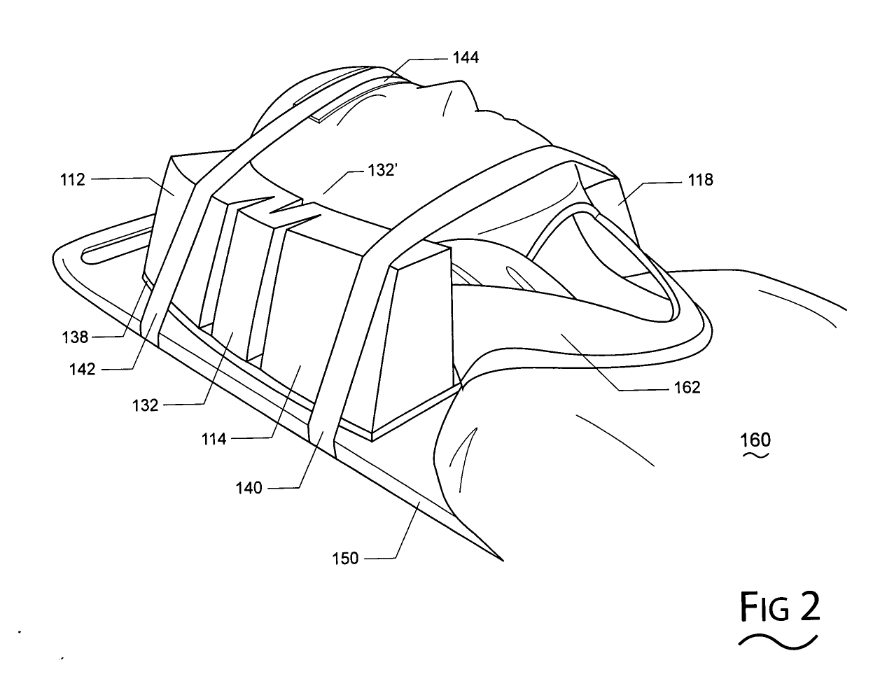

Another cervical spine immobilization device, or restraint, 110 according to the present invention is described herein with reference to the figures. As shown in FIG. 2 and FIG. 5, the restrain 110 is a used with spine board 150. The patient 160 is fitted with cervical collar 162, placed on spine board 50 and secured by a number of straps. The head is then secured to the board 150 using restraints 110 and straps 140, 142. Strap 142 may include forehead pad 144 to increase patient comfort, strap 140 may also include a pad, similar to forehead pad 144.

The restraint 110 may include a blocking section 112 for contacting and restraining the cranium of patient 160. The restraint 110 may also include blocking section 114 for contacting the patient's mandible and/or the cervical collar 162. In the event that patient 160 cannot be fitted with cervical collar 162, the blocking section 114 may contact the patient's mandible and neck. Blocking sections 112, 114 are connected to one another by an articulated link 132. Articulated link 132 may be formed in a variety of ways such as a flexible sheet or one or more cuts formed in the body of the restraint 110. A second restraint, substantially similar to or a mirror image of restraint 110 is placed opposite restraint 110. Blocking section 118 is shown in FIG. 2 in contact with the mandible of patient 160 and cervical collar 162. The restraint 110 may include a blocking section (not shown), opposite blocking section 112 and an articulated link (not shown).

The articulated link 132 may be formed in a variety of ways such as a flexible sheet adhered between the adjacent blocking sections 112, 114 or one or more cuts formed in the body of the restraint 110. FIG. 2 and FIG. 5 show W-cut 130′ formed by a single cut formed from the interior face 110a of restraint 110 and two substantially parallel cuts made from exterior surface 110d of restraint 110. Restraint 110 may also include a head pad 138 to raise the head of patient 160.

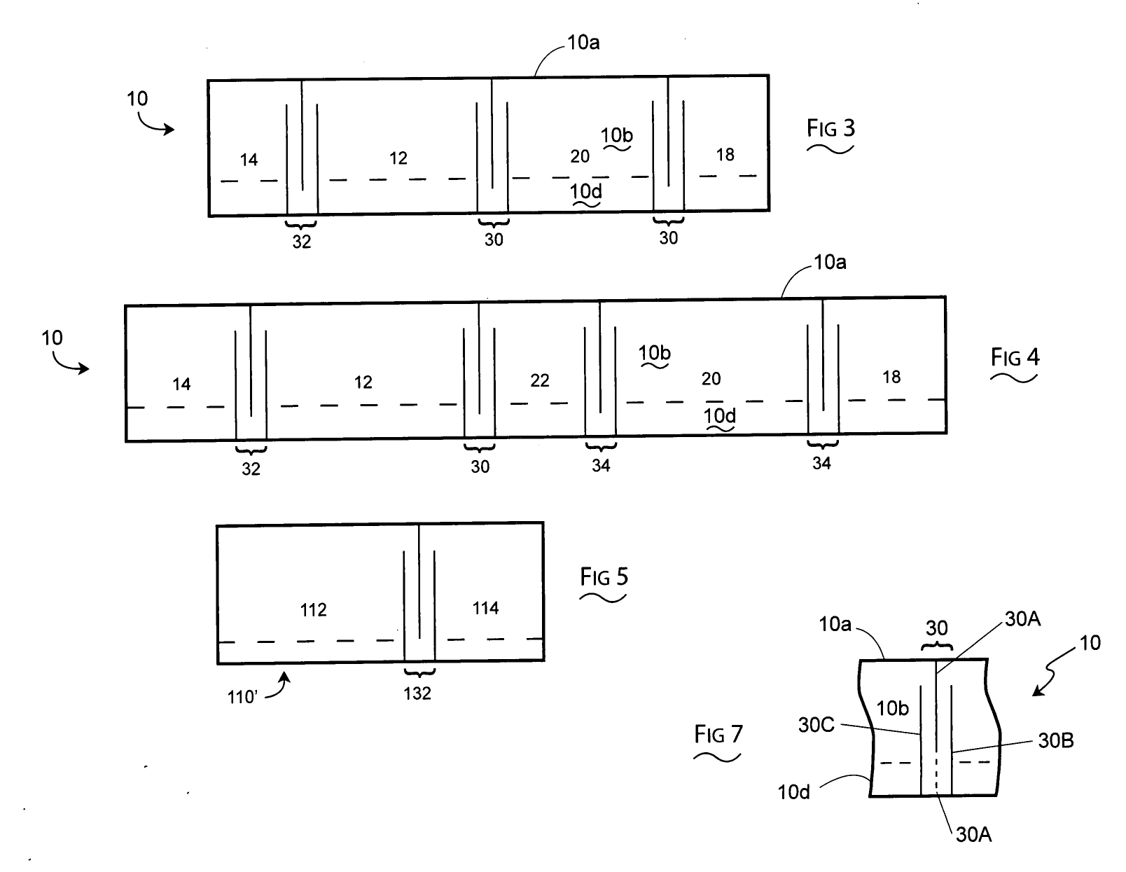

Another cervical spine immobilization device, or restraint, 10 according to the present invention is shown in FIG. 3. Similar to that shown in FIG. 4, the restrain 10 may include a blocking section 12 for contacting and restraining the cranium of patient 60, blocking section 14 for contacting the patient's mandible and/or a cervical collar. Blocking sections 12, 14 are connected to one another by an articulated link 32. The restraint 10 may include a blocking section 20, opposite blocking section 12 for contacting and restraining the cranium of patient 10 and blocking section 18, opposite blocking section 14, for contacting the patient's mandible and/or the cervical collar. Blocking sections 18, 20 are connected to one another by an articulated link 34. In contrast to the restraint of FIG. 4, the restraint of FIG. 3 does not include a central blocking section 22 but a central articulated link 30, which may be formed of a W-cut, as described with reference to FIG. 1 and FIG. 6, or may include additional cuts to allow for additional expansion and movement from link 30.

A cervical spine immobilization device or restraint 10, according to the present invention is shown in FIG. 6. As can be seen, the restrain 10 has a patient contact surface 10A, an upper surface 10B, a generally trapezoidal cross-section 10C an outer surface 10D, a base 10E and a longitudinal axis 10L. The restraint 10 includes blocking sections 12, 14, 18, 20 and 22 separated by articulated links 30, 32, 34, 36. During use, restraint 10 may be placed so that surface 10A is in contact with the head and neck of patient 10 and surface 10E is in contact with spine board 150. The head and neck are then secured to the spine board 150 by straps 40, 42. Straps 40, 42 may be formed of medical tape, a dual adhesive tape (shown in U.S. Pat. No. 5,360,393, entitled “Dual Adhesive Strap for Head Immobilization” and showing California Medical Products, Inc. as Assignee, herein incorporated by reference in its entirety) or a pre-formed tape having a pad 44 (as shown in FIG. 1) to contact the forehead, chin and/or cervical collar. Straps 40, 42 draw restraint 10 in toward the head of patient 60 to improve stability and ‘lock’ the head in position.

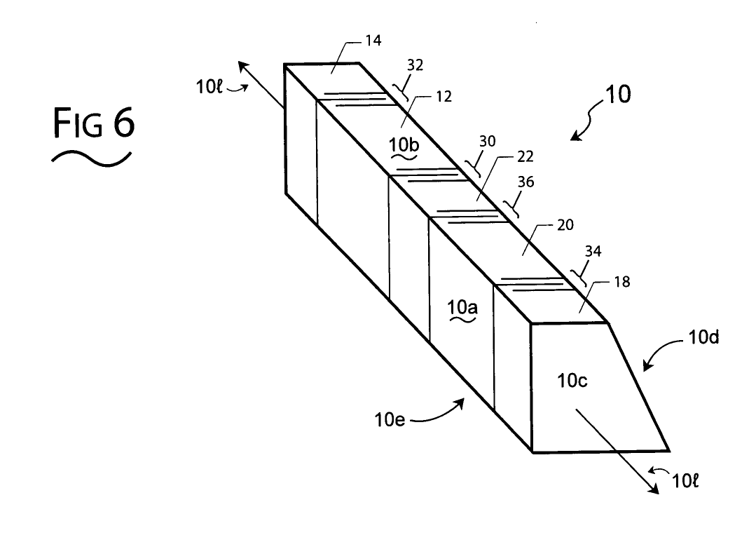

An articulated link 30, according to the present invention is shown in FIG. 7. The link 30 may be of any suitable configuration or material. One suitable construction for articulated link 30 is referred to as a W-cut in which a cut 30A is made from one side of restrain 10 while two cuts 30B, 30C are made from the opposite side of the restrain. Generally, a more flexible restraint 10 is formed when cut 30A is made from the patient contact surface 10A, and cuts 30B, 30C are made from side 10D so that cut 30A is between cuts 30B and 30C. The flexibility of articulated link 30 is increased when cut 10A is made so that it ends before contacting surface 10D and the end of cut 30A is roughly the same distance from surface 10D at upper surface 10B and lower surface 10E, resulting in an extension cut 30A′ from cut 30A that is not visible from the upper surface 10B. Improved flexibility and consistent manufacture may also be obtained when cuts 30A, 30B and 30C are parallel to one another and/or perpendicular to the longitudinal axis 10L of the restraint.

### EXAMPLES

The present invention will be better understood by reference to the following examples which are offered by way of illustration not limitation.

The material of the restraint may be any flexible and conformable material that is sufficiently rugged to survive use in emergency medical situations. One suitable material is polymer foam. Foamed polyurethane is one especially suitable material. Table 1, below shows three suitable materials available from William T. Burnett Company of Jessup, Md., USA. While polyether polyurethane foam and polyester polyurethane foams are listed below, any suitable material may be used. Materials with widespread allergies, such as latex, should be avoided.

TABLE 1

| | | | | | | Compression Force | |
|---|---|---|---|---|---|---|---|
| | | Tensile Strength | | Elongation | | 25% Deflection | |
| | Density | (KPa) | | (%) | | (kN/m2) | |
| | (Kg/M³) | Minimum | Average | Minimum | Average | Minimum | Average |
| SEN Polyether | 28.8 ± 10% | 83 | 172 | 140 | 250 | 133 N/m² | 178 N/m² |
| Polyurethane Foam | | | | | | | |
| S82N Polyester | 32.0 ± 10% | 110 | 138 | 90 | 116 | 3.8 kN/m² | 5.2 kN/m² |
| Polyurethane Foam | | | | | | | |
| S82C Polyester | 25.6 ± 10% | 117 | 172 | 80 | 120 | 4.1 kN/m² | 5.9 kN/m² |
| Polyurethane Foam | | | | | | | |

As set fourth in Table 2, a number of samples were made. Each sample included two primary blocking sections (12, 20 in FIG. 4) for contacting and restraining the cranium of patient 60 (listed as PBS, in Table 2), two secondary blocking sections (14, 18 in FIG. 4) for contacting the patient's mandible and/or the cervical collar 62 (listed as SBS, in Table 2) and a central blocking section (22 in FIG. 4) for contacting the top of the patients head (listed as CBS, in Table 2). The material and geometry of Samples 1-6 are set forth in TABLE 2.

TABLE 2

| Sam- | | | | Base | Top | | | |
|---|---|---|---|---|---|---|---|---|
| ple | Material | Height | Length | Width | Width | PBS | SBS | CBS |
| (—) | (—) | (cm) | (cm) | (cm) | (cm) | (cm) | (cm) | (cm) |
| 1 | S82N | 12.5 | 52 | 12 | 6.5 | 16 | 6.0 | 7.5 |
| 2 | S82N | 14 | 56 | 9 | 5.5 | 14 | 8.0 | 10.0 |
| 3 | S82C | 15 | 55 | 11.5 | 8 | 16 | 6.5 | 10.0 |
| 4 | S82C | 14 | 55 | 12.5 | 8.5 | 16 | 6.5 | 10.0 |
| 5 | SEN | 15.4 | 55 | 12 | 7.5 | 16 | 6.5 | 10.0 |
| 6 | SEN | 14 | 55 | 12 | 8.5 | 16 | 6 | 10.5 |

Preliminary measurements of stability were taken by placing an EMT-Paramedic on a spine board in a cervical collar (Laerdal Stifneck® Select extrication collar, available from Laerdal Medical Corporation Wappingers Falls, N.Y., USA) with straps at the nipple, navel and knee (standard EMS protocol). The cervical spine immobilization device of the present invention was applied as shown in FIG. 1, the Laerdal Headbed II head restraint (Laerdal Medical Corporation Wappingers Falls, N.Y., USA) and the Multi-Grip Head Immobilizer (available from Itek Manufacturing Winnsboro, Tex., USA) as directed by the manufacturers. Data was also collected when the patient was secured to the board using a strip of tape across the forehead and a second strip of tape across the chin and cervical collar. The movement was measured by nose movement in one direction when the patient was asked to turn his head. The patient was also asked to give a subjective rating of stability on a scale of 1 to 10. The data is shown in TABLE 3, below.

TABLE 3

| | | Stability |
|---|---|---|
| | Distance | (Subjective 1-10) |
| Restraint | (cm) | (—) |
| 1 | 1.0 | 8 |
| 2 | 1.0 | 9 |
| 3 | 1.1 | 7 |
| 4 | 1.0 | 7 |
| 5 | 1.1 | 5 |
| 6 | 1.1 | 6 |
| Headbed II | 1.9 | 6 |
| Multi-Grip | 2.5 | 4 |
| Tape Only | 1.9 | 3 |

The present invention should not be considered limited to the specific examples described herein, but rather should be understood to cover all aspects of the invention. Various modifications, equivalent processes, as well as numerous structures and devices to which the present invention may be applicable will be readily apparent to those of skill in the art. Those skilled in the art will understand that various changes may be made without departing from the scope of the invention, which is not to be considered limited to what is described in the specification.

What is claimed is:

1. A cervical spine immobilization device comprising:
a body having at least one articulated link dividing the body into a plurality of blocking sections.

2. The cervical spine immobilization device of claim 1, further comprising at least three links and at least four blocking sections.

3. The cervical spine immobilization device of claim 1, wherein the body is formed of a foamed polymeric material and comprises one link and two blocking sections.

4. The cervical spine immobilization device of claim 3, wherein the at least one articulated link is formed by one or more cuts in the body.

5. The cervical spine immobilization device of claim 1, wherein the body is formed of a foamed polymeric material and comprises at least three links and at least four blocking sections.

6. The cervical spine immobilization device of claim 1, wherein the at least one articulated link is formed by three cuts in the body.

7. The cervical spine immobilization device of claim 1, wherein the at least one articulated link is formed by W-cuts in the body.

8. The cervical spine immobilization device of claim 1, wherein the at least one articulated links allow adjacent blocking sections be adjusted to conform the angle of blocking sections to the head, jaw and neck of a patient.

9. A cervical spine immobilization system, comprising:
a first blocking section adapted to be fitted to a first side of a patients head;
a first articulated blocking section, joined to said first blocking section;
a second blocking section adapted to be fitted to a second side of a patients head; and
a second articulated blocking section, joined to said second blocking section.

10. The cervical spine immobilization system of claim 9, further comprising:
a first link section being integrally formed between said first blocking section and said first articulated blocking section; and
a second link section being integrally formed between said second blocking section and said second articulated blocking section.

11. The cervical spine immobilization system of claim 10, further comprising a third link section integrally formed between said first blocking section and said second blocking section.

12. The cervical spine immobilization system of claim 9, further comprising:
a third blocking section positioned between and linked to said first and second blocking sections.

13. The cervical spine immobilization system of claim 9, further comprising:
a third blocking section;
a third link section between said first blocking section and said third blocking section; and
a fourth link section between said third blocking section and said second blocking section.

14. The cervical spine immobilization system of claim 14, wherein
said first, second and third blocking sections, said first and second articulated blocking sections and said first, second, third, and fourth link sections are integrally formed from a polymer foam body.

15. A cervical spine immobilization device, comprising:
a body having a predetermined cross section and a longitudinal axis, the body including a base for contact with a spine board, a first surface for contact with the head of a patient and a second side positioned away from the head of a patient and a plurality of cuts in extending part of the way through said body and separating the body into a plurality of blocking sections for contact with the head or neck of a patient.

16. The cervical spine immobilization device of claim 15, wherein said plurality of cuts includes at least one cut extending from the first surface of the body and toward said second surface and at least one cut extending from the second surface of the body and toward said first surface.

17. The cervical spine immobilization device of claim 15, wherein said plurality of cuts includes at least one cut extending from the first surface of the body and toward said second surface and at least two cuts extending from the second surface of the body and toward said first surface.

18. The cervical spine immobilization device of claim 15, wherein said plurality of cuts includes at least one cut, perpendicular to the longitudinal axis, extending from the first surface of the body and toward said second surface and at least two cuts, perpendicular to the longitudinal axis, extending from the second surface of the body and toward said first surface.

19. The cervical spine immobilization device of claim 15, wherein further comprising:
a first unitary body having a first blocking section; a second blocking section and a first link formed of a plurality of cuts between said first and second blocking sections; and
a second unitary body having a third blocking section, a fourth blocking section and a second link formed of a plurality of cuts between said third and fourth blocking sections.

20. The cervical spine immobilization device of claim 19, wherein further comprising:
a third link formed of a plurality of cuts between said first unitary body and said second unitary body.
